(19) United States
(12) Patent Application Publication
(10) Pub. No.: US 2024/0148320 A1
(43) Pub. Date: May 9, 2024

(54) SYSTEMS AND WEARABLE DEVICES FOR GUIDING BREATHING OF A WEARER AND METHODS OF USING SAME

(71) Applicant: LULULEMON ATHLETICA CANADA INC., Vancouver (CA)

(72) Inventors: Navjot KAILAY, North Vancouver (CA); Ellisa Kathleen CALDER, Kelowna (CA); Sian Victoria ALLEN, Vancouver (CA); Kerem DOGURGA, Vancouver (CA); Adrian Ka Ming LAI, Coquitlam (CA); Jean-Louis IACONIS; Afshin Frederick MEHIN, Burnaby (CA); Paul Alexander Hendrik BUCCI, Vancouver (CA)

(21) Appl. No.: 18/550,938

(22) PCT Filed: Mar. 18, 2022
(86) PCT No.: PCT/CA2022/050414

§ 371 Date: Sep. 15, 2023

Related U.S. Application Data
(60) Provisional application No. 63/166,159, filed on Mar. 25, 2021.

(51) Int. Cl. A61B 5/00; A61B 90/00

(52) U.S. Cl. CPC A61B 5/486; A61B 5/7405; A61B 5/7455; A61B 90/08; A61B 2560/0406; A61B 2560/0443

(57) ABSTRACT

The disclosure provides methods, systems and devices for guiding breathing of a wearer through a breathing pattern. The system includes a wearable device with a non-rotating body and a rotating body, the rotating body configured to rotate about the non-rotating body, a position sensor coupled to the wearable device, a physiological sensor in communication with the wearer configured to detect at least one physiological parameter of the wearer, an actuator, a guiding element in communication with the actuator and configured to provide one or more guiding signals to the wearer, and a controller in communication with the actuator, the physiological sensor and the position sensor. The controller includes an input unit, a processing unit and an output unit for transmitting an output signal to the actuator to actuate the guiding element to provide one or more guiding signals to the wearer to guide the wearer through the breathing pattern.

### FIELD

This disclosure relates to wearable devices, systems and methods for guiding breathing of a wearer. In particular, the disclosure relates to wearable devices and systems for guiding breathing of a wearer through a breathing pattern and methods of using same.

### BACKGROUND

Emotional health is intimately intertwined with physical health, and with the growing complexity of life, the relationship between physiological conditions and emotional health has become of increasing interest. Stress and other emotional factors may increase the risk of disease, reduce performance and productivity, and restrict quality of life.

Physiological monitoring has been used in order to detect a person's emotional state by means of monitoring and analyzing the person's physiological response to their environment. For example, heart rate variability (HRV) has been used to derive health assessment metrics, such as overall health and wellness, fitness and stress. Based on these physiological measurements, behavioural interventions have been suggested that then help people modulate or manage their emotional health and recover from stress, which in turn may affect their physical health. Recovery from stress is important because it is during recovery that the body responds to stress and becomes prepared to perform upcoming tasks and activities.

Various interventions for recovery often include mindfulness activities, such as meditation and breathing exercises. These breathing exercises may involve breathing according to certain patterns, for example, a box pattern (also referred to as four-square breathing which involves inhaling for four counts, holding for four counts, exhaling for four counts, and holding for four counts).

Many individuals are aware of the mental and emotional benefits associated with recovery and mindfulness. Mindful practices have become more widespread through digital applications and community studios, amongst other means. However, it is often difficult for individuals to commit to longer-term practices due to a lack of guidance, tangibility of progress, and engagement due to feelings of “busyness” and a lack of time.

Thus, there exists a need for devices and systems that guide a wearer through various breathing patterns to increase recovery, stress management, anxiety relief and/or feelings of wellness.

### SUMMARY

In various embodiments, the present disclosure provides wearable devices and systems for guiding breathing of a wearer through a breathing pattern and methods of using same.

Various aspects of the present disclosure provide a system for guiding breathing of a wearer through a breathing pattern, the system comprising: a wearable device comprising a non-rotating body and a rotating body adjacent to the non-rotating body, the rotating body configured to rotate about the non-rotating body substantially frictionless or almost frictionless; a position sensor coupled to the wearable device to generate one or more position signals; a physiological sensor in communication with the wearer configured to detect at least one physiological parameter of the wearer and generate one or more physiological signals; an actuator; a guiding element in communication with the actuator and configured to provide one or more guiding signals to the wearer; and a controller in communication with the actuator, the physiological sensor and the position sensor, the controller comprising: an input unit for receiving the one or more physiological signals from the physiological sensor and the one or more position signals from the position sensor, a processing unit for determining a physiological state of the wearer based on the one or more physiological signals and the one or more position signals and generating an output signal, and an output unit for transmitting the output signal to the actuator to actuate the guiding element to provide one or more guiding signals to the wearer to guide the wearer through the breathing pattern.

Various aspects of the present disclosure provide a system for guiding breathing of a wearer through a breathing pattern, the system comprising: a wearable device; a position sensor coupled to the wearable device to generate one or more position signals; a physiological sensor in communication with the wearer configured to detect at least one physiological parameter of the wearer and generate one or more physiological signals; an actuator; a guiding element in communication with the actuator and configured to provide one or more guiding signals to the wearer; and a controller in communication with the actuator, the physiological sensor and the position sensor, the controller comprising: an input unit for receiving the one or more physiological signals from the physiological sensor and the one or more position signals from the position sensor, a processing unit for determining a physiological state of the wearer based on the one or more physiological signals and the one or more position signals and generating an output signal, and an output unit for transmitting the output signal to the actuator to actuate the guiding element to provide one or more guiding signals to the wearer to guide the wearer through the breathing pattern

According to various embodiments, the wearable device does not include a visual display.

In various embodiments, the guiding element is coupled to the non-rotating body. In other embodiments, the guiding element is coupled to the rotating body.

In various embodiments, the actuator is coupled to the non-rotating body.

In various embodiments, the one or more guiding signals are haptic signals. For example, the actuator is a linear resonant actuator, the guiding element providing one or more vibrotactile guiding signals to the wearer to guide the wearer through the breathing pattern. For example, the actuator is an eccentric rotating mass motor, the guiding element providing one or more vibrotactile guiding signals to the wearer to guide the wearer through the breathing pattern. For example, the actuator is a voice coil actuator, the guiding element providing one or more tactile guiding signals to the wearer to guide the wearer through the breathing pattern. For example, the actuator is a piezoelectric actuator in communication with at least one of the non-rotating body or the rotating body, the guiding element providing one or more vibrotactile or pressure-tactile guiding signals to the wearer to guide the wearer through the breathing pattern.

In various embodiments, the one or more guiding signals are audio feedback signals, the guiding element comprising a speaker and a microphone.

In various embodiments, the one or more guiding signals are visual signals, the guiding element comprising at least one LED.

In various embodiments, the wearable device further comprises at least one washer positioned between the non-rotating body and the rotating body to reduce friction therebetween. For example, the at least one washer may be a silicone washer.

In various embodiments, the wearable device further comprises a plurality of bearings positioned between the non-rotating body and the rotating body to reduce friction therebetween.

In various embodiments, the position sensor is coupled to the non-rotating body. In various embodiments, one or more of a plurality of components of the position sensor is coupled to the non-rotating body and one or more of the plurality of components of the position sensor is coupled to the rotating body. In various embodiments, the position sensor may be a rotary encoder.

In various embodiments, the wearable device further comprises a battery to power the actuator.

In various embodiments, the physiological sensor is positioned remotely from the wearable device.

In various embodiments, the controller is positioned remotely from the wearable device and is in communication with the wearable device by wire or wirelessly.

In various embodiments, the wearable device further comprises a switch. The switch may be for turning the system on and off. Alternatively, or in addition, the switch may be in communication with the controller and activation of the switch generates an input signal to the input unit that is processed by the processing unit to generate the output signal that is transmitted to the actuator to actuate the guiding element.

In various embodiments, the wearable device is a ring, a bracelet or a necklace.

Various aspects of the present disclosure provide a system for guiding breathing of a wearer through a breathing pattern, the system comprising: a wearable device comprising a body and a switch, the switch to generate one or more input signals; a position sensor coupled to the body to generate one or more position signals; a physiological sensor in communication with the wearer configured to detect at least one physiological parameter of the wearer and generate one or more physiological signals; an actuator; a guiding element in communication with the actuator and configured to provide one or more guiding signals to the wearer; and a controller in communication with the actuator, the physiological sensor, the position sensor and the switch, the controller comprising: an input unit for receiving the one or more physiological signals from the physiological sensor, the one or more position signals from the position sensor and the one or more input signals from the switch; a processing unit for determining a physiological state of the wearer based on the one or more physiological signals, the one or more position signals and the one or more input signals, and generating an output signal, and an output unit for transmitting the output signal to the actuator to actuate the guiding element to provide one or more guiding signals to the wearer to guide the wearer through the breathing pattern.

According to various embodiments, the wearable device does not include a visual display.

In various embodiments, the guiding element is coupled to the body.

In various embodiments, the actuator is coupled to the body.

In various embodiments, the one or more guiding signals are haptic signals. For example, the actuator is a linear resonant actuator, the guiding element providing one or more vibrotactile guiding signals to the wearer to guide the wearer through the breathing pattern. For example, the actuator is an eccentric rotating mass motor, the guiding element providing one or more vibrotactile guiding signals to the wearer to guide the wearer through the breathing pattern. For example, the actuator is a voice coil actuator, the guiding element providing one or more tactile guiding signals to the wearer to guide the wearer through the breathing pattern. For example, the actuator is a piezoelectric actuator in communication with the body, the guiding element providing one or more vibrotactile or pressure-tactile guiding signals to the wearer to guide the wearer through the breathing pattern.

In various embodiments, the one or more guiding signals are audio feedback signals, the guiding element comprising a speaker and a microphone.

In various embodiments, the one or more guiding signals are visual signals, the guiding element comprising at least one LED.

In various embodiments, the position sensor may be a rotary encoder.

In various embodiments, the wearable device further comprises a battery to power the actuator.

In various embodiments, the physiological sensor is positioned remotely from the wearable device.

In various embodiments, the controller is positioned remotely from the wearable device and is in communication with the wearable device by wire or wirelessly.

In various embodiments, the wearable device is a ring, a bracelet or a necklace.

In various embodiments, the controller is within a smart device. For example, the smart device may be an exercise smart device, a smart mirror device, a smart treadmill device, a smart stationary bicycle device, a smart home gym device, a smart weight device, a smart weightlifting device, a smart bicycle device, a smart exercise mat device, a smart rower device, a smart elliptical device, a smart vertical climber, a smart swim machine, a smart boxing gym, a smart boxing bag, a smart boxing dummy, a smart grappling dummy, a smart dance studio, a smart dance floor, a smart dance barre, a smart balance board, a smart slide board, a smart spin board, a smart ski trainer, a smart trampoline, or a smart vibration platform.

Various aspects of the present disclosure further provide methods for guiding breathing of a wearer through a pre-determined breathing pattern, the method comprising: detecting at least one physiological parameter of the wearer with a physiological sensor in communication with the wearer and providing one or more physiological signals to an input unit of a controller; detecting a position of a position sensor coupled to a wearable device and providing one or more position signals to the input unit of the controller, wherein the wearable device comprises a non-rotating body and a rotating body that rotates about a non-rotating body of the wearable device substantially frictionless or almost frictionless, the non-rotating body adjacent the wearer; processing the one or more physiological signals and the one or more position signals at a processing unit of the controller to determine a physiological state of the wearer and generate an output signal; transmitting the output signal from an output unit of the controller to an actuator; actuating a guiding element in communication with the actuator; and providing one or more guiding signals from the guiding element to the wearer of the wearable device to guide the wearer through the pre-determined breathing pattern.

Various aspects of the present disclosure further provide methods for guiding breathing of a wearer through a pre-determined breathing pattern, the method comprising: detecting at least one physiological parameter of the wearer with a physiological sensor in communication with the wearer and providing one or more physiological signals to an input unit of a controller; detecting a position of a position sensor coupled to a wearable device and providing one or more position signals to the input unit of the controller, processing the one or more physiological signals and the one or more position signals at a processing unit of the controller to determine a physiological state of the wearer and generate an output signal; transmitting the output signal from an output unit of the controller to an actuator; actuating a guiding element in communication with the actuator; and providing one or more guiding signals from the guiding element to the wearer of the wearable device to guide the wearer through the pre-determined breathing pattern.

In various embodiments, the methods further comprise actuating a switch and providing one or more input signals to the input unit of the controller and processing the one or more input signals with the one or more physiological signals and the one or more position signals at the processing unit of the controller to determine the physiological state of the wearer.

In various embodiments, the input unit receives the one or more position signals from the position sensor comprising a rotary encoder, the processing unit determining the physiological state of the wearer and generating the output signal to slow down rotation of the rotating body.

Various aspects of the present disclosure also provide a computer-readable medium having stored thereon computer program code configured when executed by one or more processors to cause the one or more processors to perform a method as described herein.

Various aspects of the present disclosure provide a wearable device for guiding breathing of a wearer through a breathing pattern, the wearable device comprising: a non-rotating body; a rotating body adjacent to the non-rotating body, the rotating body configured to rotate about the non-rotating body substantially frictionless or almost frictionless; and a position sensor coupled to the wearable device to generate one or more position signals.

In various embodiments, the wearable device further comprises a guiding element for providing one or more guiding signals to the wearer to guide breathing of the wearer through the breathing pattern. The guiding element may be coupled to the non-rotating body or to the rotating body. For example, the guiding element may be a vibrating coin motor or a pager motor.

In various embodiments, the wearable device further comprises an actuator coupled to the non-rotating body, the actuator in communication with the guiding element.

In various embodiments, the position sensor is coupled to the non-rotating body. In various embodiments, one or more of a plurality of components of the position sensor is coupled to the non-rotating body and one or more of the plurality of components of the position sensor is coupled to the rotating body. In various embodiments, the position sensor may be a rotary encoder.

In various embodiments, the wearable device further comprises a switch for activating the guiding element and/or for turning the wearable device on and off. The switch may be in communication with the controller and activation of the switch generates an input signal to the input unit that is processed by the processing unit to generate the output signal that is transmitted to the actuator to actuate the guiding element. For example, the switch may be coupled to the guiding element or to the rotating body.

In various embodiments, the wearable device further comprises a power source, such as a battery.

Other aspects and features of the present invention will become apparent to those of ordinary skill in the art upon review of the following description of specific embodiments of the invention in conjunction with the accompanying claims.

### BRIEF DESCRIPTION OF THE DRAWINGS

In drawings which illustrate embodiments of the disclosure,

FIG. 1 is a block diagram of a system for guiding breathing of a wearer through a breathing pattern in accordance with an embodiment of the invention.

FIG. 2 is a plan view of an example of a prototype of a system for guiding breathing of a wearer through a breathing pattern.

FIG. 3 shows a block diagram of a method for guiding breathing of a wearer through a pre-determined breathing pattern in accordance with an embodiment of the invention.

FIG. 4 shows a method of providing one or more guiding signals from the guiding element to the wearer of the wearable device to guide the wearer through the breathing pattern in accordance with an embodiment of the invention.

FIG. 5A is a cross-sectional view of an example of a system for guiding breathing of a wearer through a breathing pattern in accordance with an embodiment of the invention. FIG. 5B is a perspective view of the system of FIG. 5A.

FIG. 6 shows a method of providing one or more guiding signals from the guiding element to the wearer of the wearable device to guide the wearer through the breathing pattern in accordance with another embodiment of the invention.

FIGS. 7A to 7I show wearable devices in accordance with various embodiments of the invention with different configurations for the switch and guiding element. FIG. 7A is a right-side diagram view of a wearable device in accordance with an embodiment of the invention. FIG. 7B is a left-side plan view of the wearable device of FIG. 7A. FIG. 7C is a cross-sectional side view of the wearable device of FIG. 7A.

FIG. 7D is a right-side diagram view of a wearable device in accordance with another embodiment of the invention. FIG. 7E is a left-side plan view of a wearable device of FIG. 7D. FIG. 7F is a right-side diagram view of a wearable device in accordance with a further embodiment of the invention. FIG. 7G is a left-side plan view of the wearable device of FIG. 7F. FIG. 7H is a right-side diagram view of a wearable device in accordance with a further embodiment of the invention. FIG. 7I is a left-side plan view of the wearable device of FIG. 7H.

FIG. 8 shows a method of providing one or more guiding signals from the guiding element to the wearer of the wearable device to guide the wearer through the breathing pattern in accordance with a further embodiment of the invention.

FIG. 9 shows a method of providing one or more guiding signals from the guiding element to the wearer of the wearable device to guide the wearer through the breathing pattern in accordance with a further embodiment of the invention.

FIG. 10 shows a method of providing one or more guiding signals from the guiding element to the wearer of the wearable device to guide the wearer through the breathing pattern in accordance with a further embodiment of the invention.

FIG. 11 shows a method of providing one or more guiding signals from the guiding element to the wearer of the wearable device to guide the wearer through the breathing pattern in accordance with a further embodiment of the invention.

FIG. 12 shows the wearable device to guide the wearer through the breathing pattern within a smart system in accordance with an embodiment of the invention.

### DETAILED DESCRIPTION

In the context of the present disclosure, various terms are used in accordance with what is understood to be the ordinary meaning of those terms.

Disclosed embodiments include wearable devices, systems, methods and storage media associated with guiding breathing of a wearer through a breathing pattern. In various embodiments, the disclosure provides a wearable device and system for guiding breathing of a wearer through a breathing pattern and methods of using the wearable devices and systems for executing a breathing pattern, such as a paced breathing pattern. Paced breathing is a known and validated means of eliciting recovery responses in individuals.

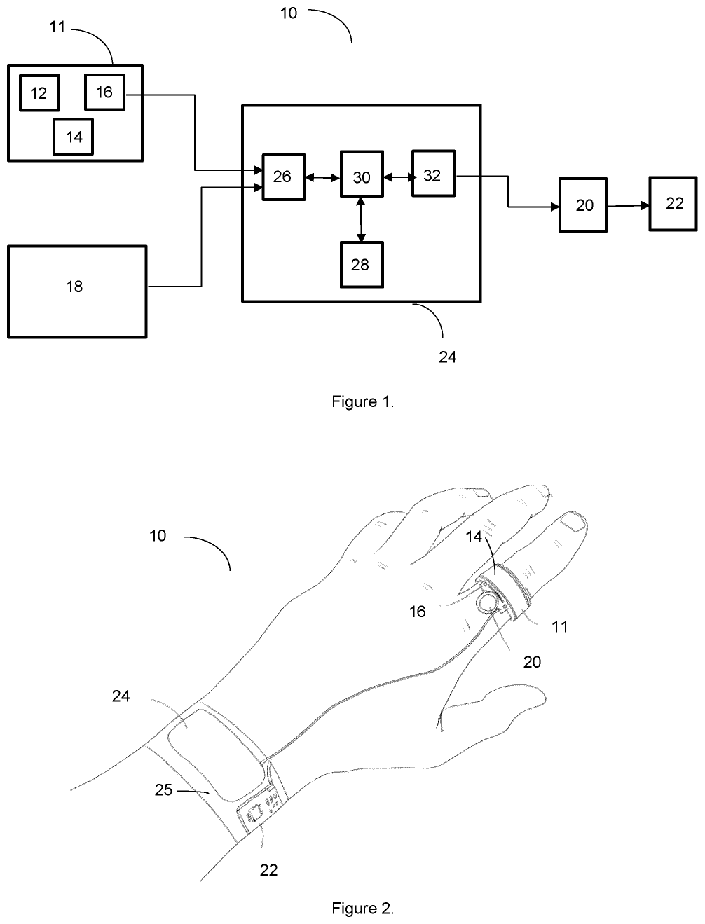

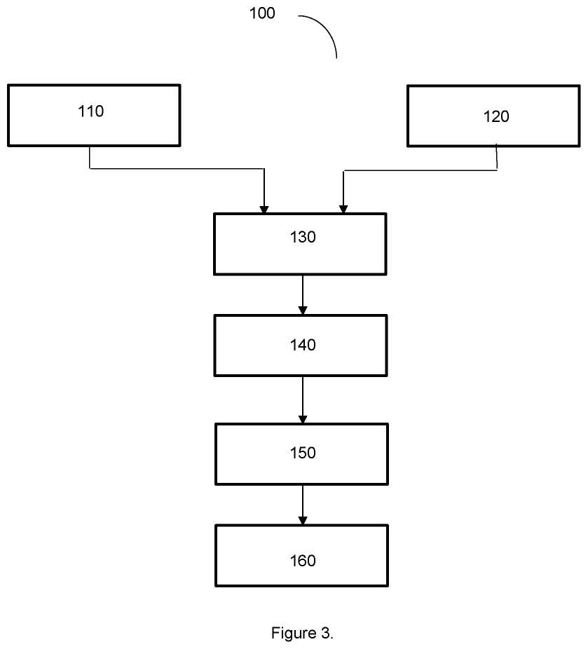

Referring to FIG. 1 and according to a first embodiment of the invention, a system 10 for guiding breathing of a wearer through a breathing pattern is shown. The system 10 comprises a wearable device 11. The wearable device 11 comprises a non-rotating body 12 and a rotating body 14. The rotating body 14 is adjacent to the non-rotating body 12 and is configured to rotate about the non-rotating body 12 substantially frictionless or almost frictionless. The wearable device 11 may also comprise at least one washer positioned between the non-rotating body 12 and the rotating body 14 in order to reduce friction therebetween. For example, the at least one washer may be a silicone washer. Alternatively, the wearable device 11 may also comprise a plurality of bearings positioned between the non-rotating body 12 and the rotating body 14 in order to reduce friction therebetween. The non-rotating body 12 may be adjacent the wearer and may be a ring (as shown, for example, in FIG. 2). In various embodiments, the wearable device 11 may also be, for example, a bracelet or a necklace. In various embodiments, and as discussed below, the wearable device may comprise a body, a position sensor and a switch.

In various embodiments, the rotating body 14 may be used as a “fidget” device and the wearer may rotate the rotating body 14 around the non-rotating body 12 as desired. The use of the rotating body 14 in this manner may support recovery by inducing a physiological and/or psychological relaxation response or increase present moment awareness and/or focus. The relative rotation of the bodies of the wearable device 11 provides for the benefits of fidgeting without the extra distractions created by irregular product features such as, for example, different temperature elements or unbalanced weight distribution. Use of the wearable device 11 also does not disturb others through sounds or obvious movements and can more easily be engaged as it is worn on the body of the wearer. Rotating the rotatable body 14 may draw on coordination skills with a physical demand and a small but substantial level of technical difficulty or challenge in order to assist in enhancing attention, rather than overburdening attention.

The system 10 further includes a position sensor 16 that may be coupled to the non-rotating body 12 operable to generate data indicative of movement of the rotating body 14 as one or more position signals. In various embodiments, part of the position sensor 16 may be coupled to the non-rotating body 12 while another part of the position sensor 16 may be coupled to the rotating body 14. For example, a sensing mechanism, such as an electronics circuit, controller and power source, may be coupled to the non-rotating body 12 while a sensor, such as, for example, a metal strip, magnet or LED, is coupled to the rotating body 14. The position sensor 16 may be any position sensor as would be known to a person of ordinary skill in the art, such as, for example, a rotary encoder. In various embodiments, fidgeting the rotating body 14 in one direction triggers the position sensor 16 to generate the one or more position signals while fidgeting the rotating body 14 in the other direction does not. Thus, the system 10 provides an integrated device such that the wearer may choose to aid recovery through breathing according to a breathing pattern, through fidgeting, or both.

The system 10 also includes a physiological sensor 18 in communication with the wearer and operable to generate data indicative of at least one physiological parameter of the wearer as one or more physiological signals. For example, the at least one physiological parameter of the wearer may be heart rate or heart rate variability. In other embodiments, the at least one physiological parameter of the wearer may be breathing rate, blood pressure, blood oxygen concentration, blood glucose level, skin temperature, skin wettedness, galvanic skin response, or level of activity or movement.

The system 10 further comprises an actuator 20 and a guiding element 22 in communication with the actuator 20. The guiding element 22 is operable to provide the wearer with one or more guiding signals. Such signals may be, for example, haptic (or vibrational), visual (such as a light or an image) or audio. The guiding element 22 may be located on or coupled to the non-rotating body 12, the rotating body 14 or separate from the non-rotating body 12 and rotating body 14. In various embodiments, the actuator 20 may be located on or coupled to the non-rotating body 12.

The actuator 20, the physiological sensor 18 and the position sensor 16 are all operatively connected to and in communication with a controller 24. The controller 24 may be separate from the non-rotating body 12 and the rotating body 14, the position sensor 16, the physiological sensor 18, the actuator 20 and the guiding element 22. Alternatively, the physiological sensor 18, the actuator 20, the guiding element 22 and the controller 24 may be within the same unit. In further embodiments, the physiological sensor 18 and the controller 24 may be within the same unit. In various embodiments, the wearable device 11 comprises the non-rotating body 12, the rotating body 14, the position sensor 16, the actuator 20 and the guiding element 22 and the physiological sensor 18 and the controller 24 are part of the same or different units. An example of a prototype of a system 10 is shown in FIG. 2. The system 10 comprises a wearable device 11 shown as a ring. The wearable device 11 comprises the non-rotating body 12 (not shown), the rotating body 14 and the position sensor 16. The wearable device 11 is coupled to the actuator 20. The controller 24 is part of a watch 25 that also includes the physiological sensor 18 (not shown) and the guiding element 22.

The controller 24 comprises an input unit 26, a memory 28, a processing unit 30 and an output unit 32. The input unit 26 is configured to receive data signals from the position sensor 16 as the one or more position signals, and from the physiological sensor 18 as the one or more physiological signals. The input unit 26 includes software and hardware configured to facilitate communications with the position sensor 16 and the physiological sensor 18 to the processing unit 30. The processing unit 30 may be any of various processors as will be recognized by those of ordinary skill in the art and is configured to receive data signals from the input unit 26 and the memory 28, and process such signals. Typical processing may include determining a physiological state of the wearer and comparing the physiological state to data in the memory 28 to generate an output signal. The output signal may include data to guide the wearer through a breathing pattern such as, for example, when and for how long to inhale, when and for how long to hold, and when and for how long to exhale. Furthermore, the data may be processed into different forms and formats, depending on how the guiding signals will be provided to the wearer. As understood by a person of ordinary skill in the art, a “processor” or “processing unit” as used herein includes any hardware system, hardware mechanism or hardware component that processes data, signals or other information. A processing unit can include a system with a central processing unit, multiple processing units, dedicated circuitry for achieving functionality, or other systems.

In various embodiments, the processing unit 30 may use SPI to send data between the input unit 26, the memory 28, the output unit 32 and the processing unit 30. The processing unit 30 is connected to the memory 28 and the output unit 32 and delivers processed data to one or both of the memory 28 and the output unit 32.

The memory 28 is configured to store information, including both data and instructions. The data generally include position data and physiological data that may be retrieved from the processing unit 30 along with other data that may be ancillary to the basic operation of the controller 24 and any applications retained on the controller 24. The instructions which are stored at the memory 28 generally include firmware and/or other software for execution by the processing unit 30, such as a program that controls the processing of the one or more position signals from the position sensor 16, a program that controls the processing of the one or more physiological signals from the physiological sensor 18, a program that controls the processing of one or more input signals from a switch (discussed below), a program that controls the settings of the controller 24, a program that controls various applications on the controller 24, a program that determines the physiological state of the wearer, a program that compares the physiological state of the wearer to data in the memory 28, a program that controls the transmission and reception of data and information via the input unit 26, a program that controls the generation of the output signal, a program that controls the transmission and delivery of information to the output unit 32, as well as any of various other programs that may be associated with the system 10. In various embodiments, two or more of the foregoing may be combined into one program. The instructions stored in the memory 28 for execution by the processing unit 30 may include, for example, an application (or “app”) for recommending the wearer to undertake a breathing pattern in response to the physiological state of the wearer or in response to the wearer adjusting the position sensor or switch to provide an input to the input unit 26.

The memory 28 may be of any type capable of storing information accessible by the processing unit 30, such as a memory card, ROM, RAM, write-capable memories, read-only memories, or other computer-readable media. The data may also be formatted in any computer-readable format such as, but not limited to, binary values, ASCII or Unicode.

In various embodiments, the input unit 26 of the controller 24 may communicate with each of the position sensor 16 and the physiological sensor 18 either through a wire or wirelessly. For example, for wireless communication, the input unit 26 may comprise a RF transmitter and receiver configured to transmit and receive communications signals over a short range using a wireless communications technology, such as Bluetooth®, using any of various communications protocols. Such transceivers are well known to a person of ordinary skill in the art. The input unit 26 may be configured to communicate with the position sensor 16 and the physiological sensor 18 when the input unit 26 is within a given range of the position sensor 16 and the physiological sensor 18, and transmit data from the one or more position signals and the one or more physiological signals to the processing unit 30.

Likewise, in various embodiments, the output unit 32 of the controller 24 may communicate with the actuator 20 either through a wire or wirelessly. For example, for wireless communication, the output unit 32 may comprise a RF transmitter and receiver configured to transmit and receive communications signals over a short range using a wireless communications technology, such as Bluetooth®, using any of various communications protocols. Such transceivers are well known to a person of ordinary skill in the art. The output unit 32 may be configured to communicate with the actuator 20 and transmit data from the processing unit 30 to the actuator 20. The actuator 20 then communicates with the guiding element 22 to provide one or more guiding signals to the wearer to guide the wearer through the breathing pattern.

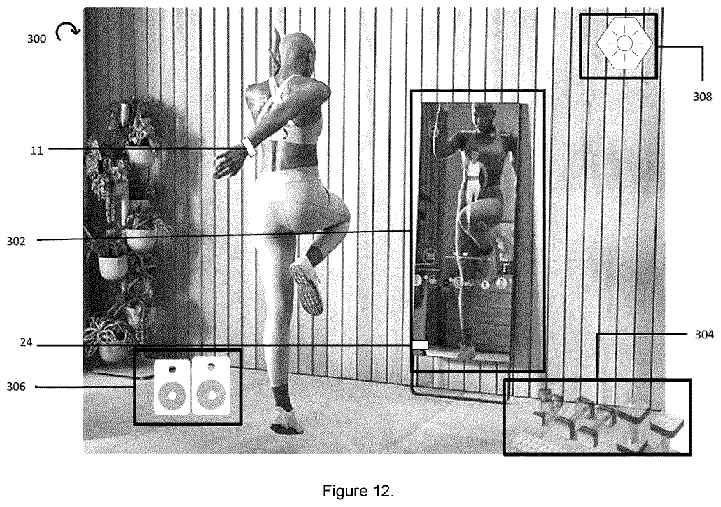

For example, FIG. 12 shows the wearable device 11 to guide the wearer through the breathing pattern within a smart system 300. Within smart system 300, the wearable device 11 communicates with a smart device 302, which comprises the controller 24. In the embodiment shown in FIG. 12, the smart device 302 is a smart mirror device. However, the smart device 302 may be any other smart device as would be known to a person of ordinary skill in the art, such as, for example, a smart exercise device, a smart treadmill device, a smart stationary bicycle device, a smart home gym device, a smart weight device, a smart weightlifting device, a smart bicycle device, a smart exercise mat device, a smart rower device, a smart elliptical device, a smart vertical climber, a smart swim machine, a smart boxing gym, a smart boxing bag, a smart boxing dummy, a smart grappling dummy, a smart dance studio, a smart dance floor, a smart dance barre, a smart balance board, a smart slide board, a smart spin board, a smart ski trainer, a smart trampoline or a smart vibration platform. Smart system 300 may also comprise various smart devices such as, for example, smart hand weights 304, smart speakers 306, and/or smart lighting 308.

Power sources are configured to provide power to one or more of the controller 24, the position sensor 16, the physiological sensor 18, the actuator 20 and the guiding element 22 during use. In various embodiments, the power source is a battery such as a rechargeable battery.

In various embodiments, the controller 24 may be any type of portable or other personal electronic device such as a smartphone, tablet computer, laptop computer, smartwatch, or any of various other mobile computing devices, or a standalone device such as a desktop PC. As will be recognized by one of ordinary skill in the art, the components of the controller may vary depending on the type of device used.

In at least one embodiment, portions of the systems and methods described herein may be implemented in suitable software code that may reside within the memory 28. Such software code may be present on the processing unit 30 at the time of manufacture or may be downloaded thereto via well-known mechanisms. A computer program product implementing an embodiment disclosed herein may therefore comprise one or more computer-readable storage media storing computer instructions translatable by a processor or microprocessor to provide an embodiment of a system or perform an embodiment of a method disclosed herein. Computer instructions may be provided by lines of code in any of various languages as will be recognized by those of ordinary skill in the art. A “computer-readable medium” may be any type of data storage medium that can store computer instructions, including, but not limited to, the memory devices discussed above.

The transmission of data from the physiological sensor 18 may occur automatically without the wearer needing to prompt the transmission. For example, some mechanism, such as a switch, may be used to turn on the physiological sensor 18 or otherwise indicate that automatic transmissions should begin. In another embodiment, the physiological sensor 18 may be configured to begin transmissions once the wearer activates it. In yet another embodiment, data transmission may occur periodically at predetermined intervals of time.

The transmission of data from the position sensor 16 occurs as a result of the wearer moving the rotating body 14 relative to the non-rotating body 12 in a particular direction, indicating that the user would like to be guided through a breathing pattern. In other embodiments, the transmission of data from the position sensor 16 occurs as a result of movement of the position sensor 18, regardless of its location on the non-rotating body 12 or between the non-rotating body and the rotating body 14.

In various embodiments, the memory 28 is configured to store data of a plurality of known breathing patterns in a breathing pattern database. The at least one physiological parameter can be measured, the wearer may move the position sensor 18 in a certain direction or way, or the wearer may provide an input signal to the input unit 26 (such as, for example, by turning on a switch), and such data is processed to determine a physiological state of the wearer or that the wearer would like to breath according to a breathing pattern. Based on the physiological state of the wearer, the processing unit 30 may determine whether the wearer would likely benefit from breathing according to a breathing pattern. Such processing may comprise comparing the physiological state to data in the memory 28. Computer instructions can also be provided by lines of code in any of various languages as will be recognized by those of ordinary skill in the art.

For example, if the at least one physiological parameter is heart rate or heart rate variability, and these values for the wearer are above a certain threshold value, then recovery of the wearer from that stress may be aided by breathing according to a certain breathing pattern, which is stored in the breathing pattern database. “Threshold level” refers to a range of values for the at least one physiological parameter, above which a wearer is considered to be in a state of stress and could benefit from the effects of breathing according to a breathing pattern, based on a population of individuals for a particular physiological parameter. The threshold level can be determined based on measurements of a population of wearers for a physiological parameter and whether they consider themselves to be in a state of stress.

Thus, the data obtained from the position sensor 16 and the physiological sensor 18 may be processed to determine the physiological state of the wearer and then the data of the physiological state is compared to the data included in the breathing pattern database in order to determine whether the wearer would be more likely than not to follow a breathing pattern in order to aid in recovery from stress and/or which breathing pattern to follow.

The data may be processed using the software application or “app” stored in a computer readable medium such as the memory 28 of the controller 24. The processing unit 30 of the controller 24 is configured to process the instructions for the app. The processing unit 30 may be controlled by computer-executable instructions stored in the memory 28 so as to provide functionality as is described herein. For example, the processing unit 30 may process the physiological or position data in order to present a recommendation to the wearer via the guiding element to undertake a breathing pattern, in a format for quick and easy communication to the wearer.

In various embodiments, a non-transient computer readable medium contains instructions for controlling the guiding element 22 via the actuator 20 by receiving the output signal from the processing unit 30 and presenting a signal or recommendation to the wearer to breath according to a breathing pattern. The guiding element 22 then provides one or more guiding signals to the wearer to guide the wearer through the breathing pattern.

In various embodiments, the wearable device does not include a visual display, such as a screen. By not including such a visual distraction, the wearable device may assist in promoting focus on the beneficial effects of using the fidget functionality of the wearable device or breathing according to the breathing pattern.

In various embodiments, the one or more guiding signals are haptic signals. The actuator 20 may then be a linear resonant actuator, an eccentric rotating mass motor or a piezoelectric actuator in communication with at least one of the non-rotating body 12 or the rotating body 14, the guiding element 22 providing one or more vibrotactile guiding signals to the wearer to guide the wearer through the breathing pattern. The actuator 20 may also be a voice coil actuator, the guiding element 22 providing one or more tactile guiding signals to the wearer to guide the wearer through the breathing pattern.

In various embodiments, the one or more guiding signals are audio feedback signals. The guiding element may then comprise a speaker and a microphone.

In various embodiments, the one or more guiding signals are visual signals. The guiding element 22 may then comprise at least one LED.

The guiding element 22 includes software and hardware configured to facilitate communications with the actuator 20 to the wearer. The hardware may include a display screen configured to visually display graphics, text and other data to the user to guide the user through a breathing pattern. The guiding element 22 may also include a microphone and/or speakers to facilitate audio communications with the user.

In various embodiments, the methods disclosed herein provide a customized recommendation for breathing patterns to aid in recovery from stress, either based on measurements of at least one physiological parameter or based on movement of the position sensor as initiated by the wearer or other input signal provided by the wearer. The methods disclosed herein are based on an optimized use of breathing patterns to aid in the recovery from stress, coupled with a device that provides the recovery benefits of fidgeting.

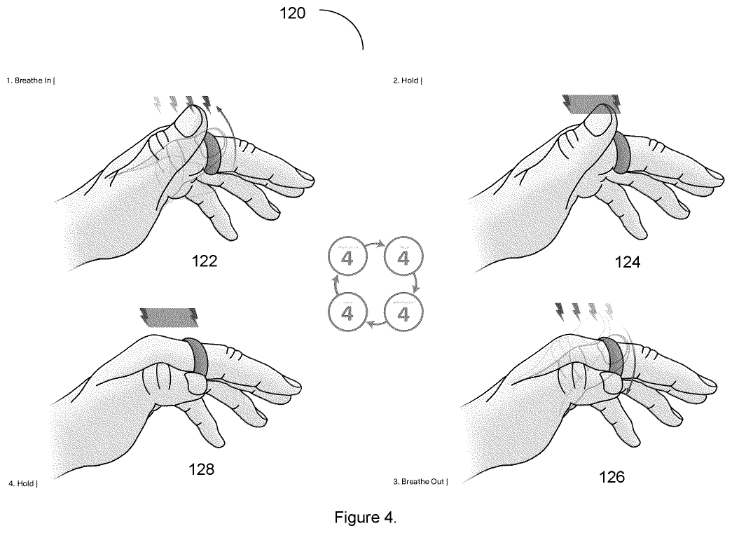

An exemplary embodiment of the methods disclosed herein is shown in FIG. 3. In various embodiments of a method 100 for guiding breathing of a wearer through a breathing pattern, the method includes detecting at least one physiological parameter of the wearer with a physiological sensor in communication with the wearer and providing one or more physiological signals to an input of a processing unit of a controller 110. The method further includes detecting a position of a position sensor coupled to a wearable device, the wearable device comprising a rotating body and providing one or more position signals to the input unit of the processing unit of the controller 120. The rotating body of the wearable device rotates about a non-rotating body of the wearable device substantially frictionless or almost frictionless with the non-rotating body adjacent to the user.

The method further includes processing the one or more physiological signals and the one or more position signals at the processing unit of the controller to determine a physiological state of the wearer 130 and generating an output signal or recommendation for a breathing pattern for the wearer to follow 140. If the wearer has rotated the position sensor in a particular direction, the processing unit will determine that the wearer has chosen to undergo a breathing pattern and is in a physiological state that may benefit from breathing according to a breathing pattern, and an output signal to the actuator and guiding element are generated. If the at least one physiological parameter is above a threshold level as stored in a memory of the controller, the processing unit will determine a physiological state of the wearer indicating that the wearer may be in a state of stress and may benefit from using a breathing pattern. The processing unit will then generate an output signal for the actuator and guiding element providing guidance or recommendation to the wearer for the breathing pattern.

The output signal is then transmitted from the output unit of the controller to the actuator, which in turn actuates the guiding element 150. The wearer is then provided one or more guiding signals to follow in order to breathe according to a breathing pattern 160. In various embodiments, the one or more guiding signals to the wearer may be to slow down rotation of the rotating body on the basis that intense, continuous rotation of the rotating body may be indicative of a higher stress physiological state of the wearer. In various embodiments, if the physiological parameter of the user goes below the threshold value, the controller may then cease providing the output signal.

By providing guiding signals to the wearer in response to physical indicators, the wearer may be encouraged to engage in mindfulness behaviour, such as breathing exercises or breathing patterns, and feel calmer, more relaxed and/or more focused in response.

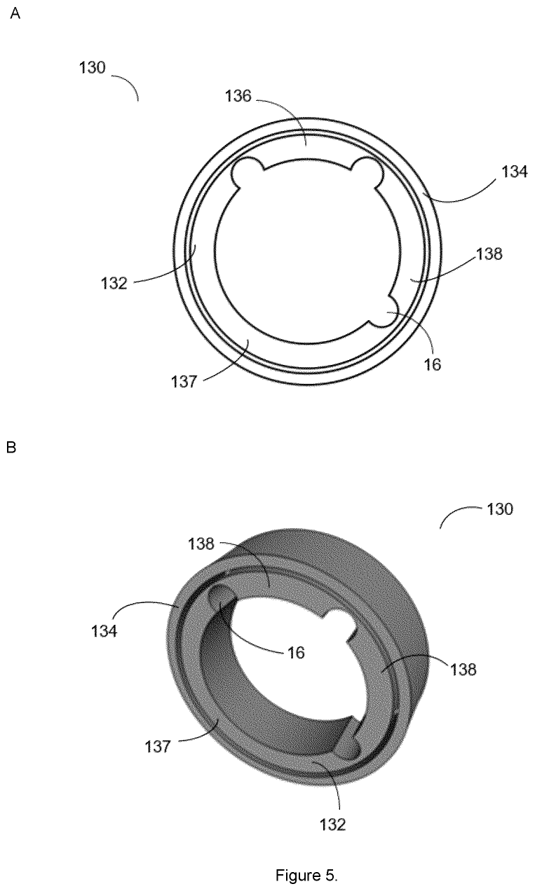

An example of a method 120 for providing one or more guiding signals to the wearer according to an embodiment of the disclosure is shown in FIG. 4. Rotation of the rotating body relative to the non-rotating body in a particular direction initiates the system to providing one or more guiding signals to the wearer to breath according to a breathing pattern 122. The one or more guiding signals may be haptic signals such that the wearer feels a vibration, signaling the wearer to inhale. When the vibration stops or changes, the wearer holds their breath 124. The wearer may then fidget the rotating body 14 in an opposite direction, resulting in further vibration signals guiding the wearer to exhale 126. Lastly, when the vibration signals stop or change, the wearer is guided to hold their breath prior to initiating the cycle again 128.

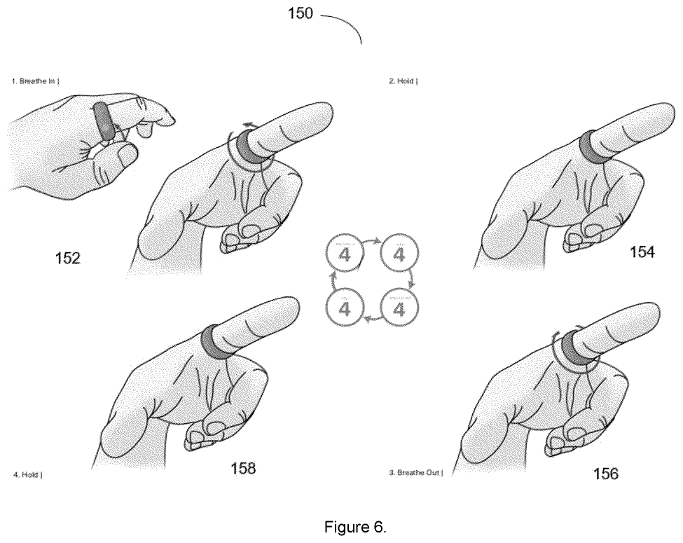

A wearable device 130 for use in this method is shown in FIGS. 5A and 5B. The wearable device 130 comprises a non-rotating body 132, a rotating body 134, a guiding element positioned within a first area 136 and a power source positioned within a second area 137 (such as, for example, a battery). The wearable device 130 further comprises one or more position sensors 16, a controller 24 and an actuator 20 positioned within a third area 138. FIG. 5B shows a perspective view of the wearable device 130 of FIG. 5A.

In various embodiments, the wearable device 130 can also comprises a switch (not shown) configured to turn on/off the device 130 to save energy by preventing drainage of the power source. In various embodiments, the switch may be in communication with the controller 24 which can be positioned remotely from the wearable device 130. When the wearer presses the switch, one or more input signals are generated. The input unit 26 of the controller 24 receives the one or more input signals from the switch and the processing unit 30 generates the output signal. The output unit 32 then transmits the output signal to the actuator 20 to actuate the guiding element to provide one or more guiding signals to the wearer to guide the wearer through the breathing pattern. The guiding element may be a vibrating coil motor and the one or more guiding signals are haptic signals to guide the wearer through the breathing pattern.

In various embodiments, the wearable device 130 may not include a position sensor as the system may be initiated through use of a switch and the wearer may spin the rotating body 134 in either direction as a fidget device. However, other embodiments of the wearable device 130 may include the position sensor 16 so that the controller 24 receives feedback as to whether the wearer is following the breathing pattern. For example, feedback indicating that the wearer is following the breathing pattern is that the wearer is not moving the wearable device 130 or keeping the wearable device 130 still. In other embodiments, as described above, where the rotating body 134 includes one or more position sensors 16, rotation of the rotating body 134 in one direction generates one or more position signals to the controller 24. The wearer then receives one or more guiding signals from the guiding element 136 guiding the wearer through the breathing pattern.

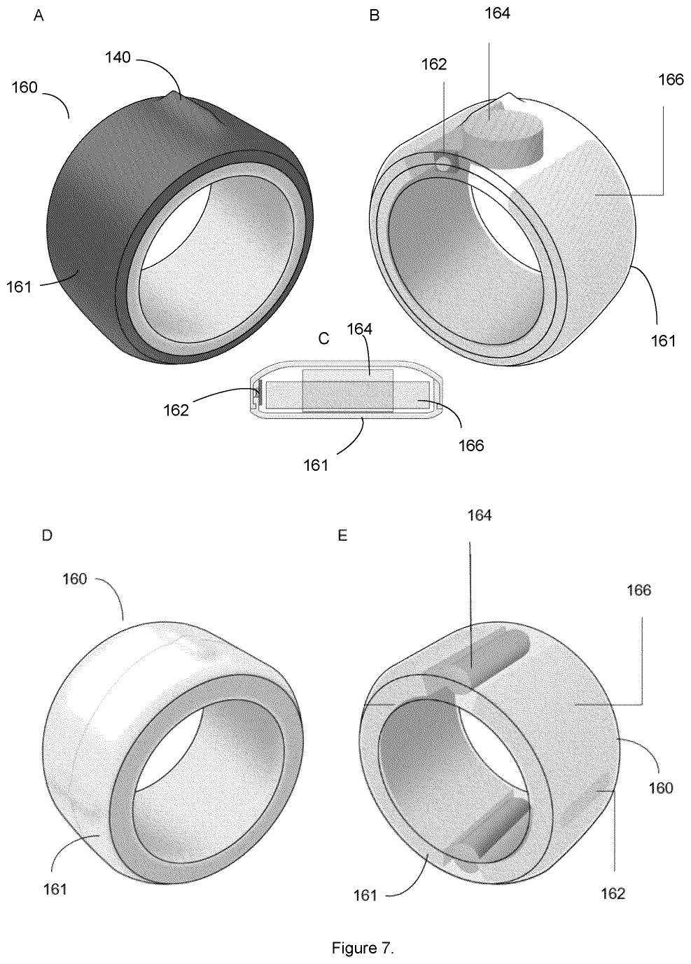

A further example of a method 150 of providing one or more guiding signals to the wearer according to an embodiment of the disclosure is shown in FIG. 6. The wearable device illustrated in FIG. 6 comprises a switch 151 configured to turn on/off the device.

As shown in FIG. 6, pressing and releasing the switch 162 or touch area begins the breathing pattern and the wearer receives one or more guiding signals to breath in 152. When the vibration stops or changes, the wearer holds their breath 154. The guiding element then provides further vibration signals, guiding the wearer to exhale 156. When the guiding signals stop or change, the wearer is guided to hold their breath prior to initiating the cycle again 158. The wearer may stop the guided breathing session by pressing on the switch or touch area 162.

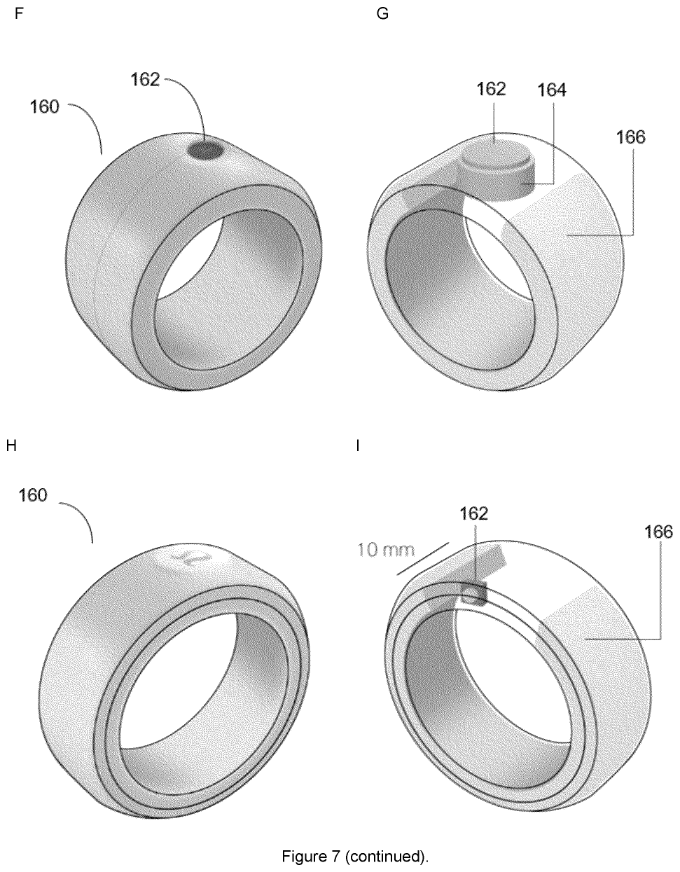

FIGS. 7A-7I illustrate a number of different configurations of a wearable device 160 in which the rotating body is omitted. The wearable device 160 comprises a body 161, a switch or touch area 162, a guiding element 164, a power source 166 (such as, for example, a battery), and optionally an actuator. The switch 162 may be in communication with the controller 24 which is positioned remotely from the wearable device 160. As indicated in FIGS. 7A-7E, the switch (or touch area) 162 can be positioned away from the guiding element 164 while in FIGS. 7F-7G the switch 162 is adjacent to the guiding element 164. The guiding element 164 can be a page motor (such as shown in FIGS. 7D and 7E) or a vibrating coil motor (such as shown in FIGS. 7A-7C) and the one or more guiding signals are haptic signals to guide the wearer through the breathing pattern. The wearable device 160 of FIGS. 7A and 7B can also include a ridge 140. One or more ridges 140 may be positioned around an exterior surface of the wearable device 160 in order to provide tactile sensory information of the wearer. FIGS. 7H and 7I show the wearable device 160 in which the guiding element 164 is positioned remotely from the wearable device 160. For example, the guiding element 164 can be positioned on a smart watch (see, for example, FIG. 12) and can be in communication with the controller 24 and the wearable device 160 wirelessly by, for example, Bluetooth®.

When the wearer presses the switch or touch area 162, one or more input signals are generated. The input unit 26 of the controller 24 receives the one or more input signals from the switch 162 and the processing unit 30 generates the output signal. The output unit 32 then transmits the output signal to the actuator 20 to actuate the guiding element 164 to provide one or more guiding signals to the wearer to guide the wearer through the breathing pattern. The wearable device 160 of FIG. 7 does not include a position sensor 16 as the system is initiated through use of the switch 162. However, other embodiments of the wearable device 160 may include the position sensor 16 so that the controller 24 receives feedback as to whether the wearer is following the breathing pattern. For example, feedback indicating that the wearer is following the breathing pattern is that the wearer is not moving the wearable device 160 or keeping the wearable device 160 still.

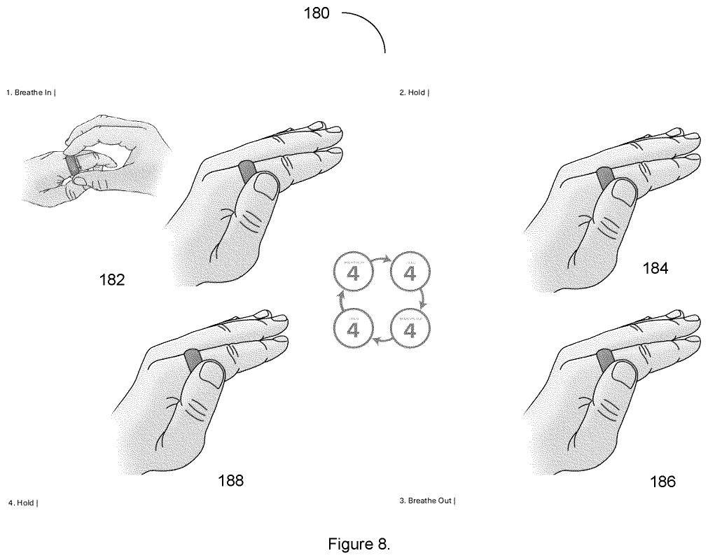

A further example of a method 180 of providing one or more guiding signals to the wearer according to a further embodiment of the disclosure is shown in FIG. 8. This embodiment is similar to that shown in FIG. 6, except that the wearer may rotate the wearable device 160 to adjust the position of the switch 162 such that the wearer can maintain pressure on the switch 162 for as long as the wearer would like to breathe according to the breathing pattern. In various embodiments, the switch 162 may comprise a gemstone (see FIG. 7F). As shown in FIG. 8, pressing the switch 162 begins the breathing pattern and the wearer receives one or more guiding signals to breath in 182. The guiding element 164 may be a vibrating coin motor as shown in FIGS. 7B, 7C and 7G, and the vibrating coin motor provides haptic signals such as vibrations, signaling the wearer to inhale. When the vibration stops or changes, the wearer holds their breath 184. The guiding element 164 then provides further vibration signals, guiding the wearer to exhale 186. When the guiding signals stop or change, the wearer is guided to hold their breath prior to initiating the cycle again 188. The wearer may stop the guided breathing session by releasing the switch 162.

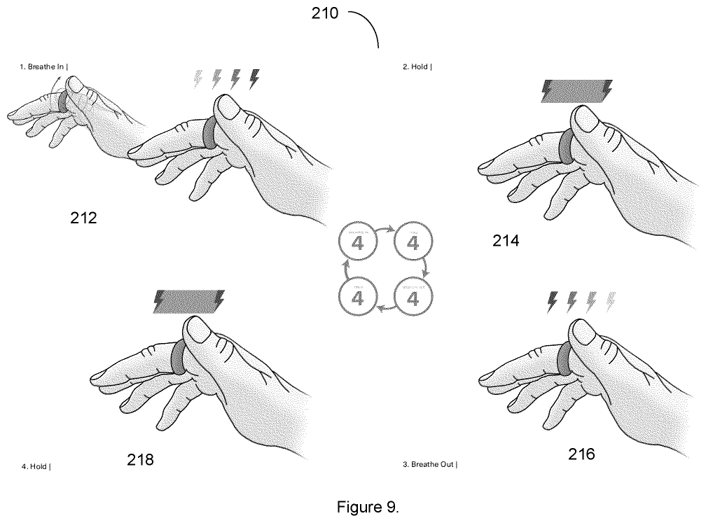

An additional example of a method 210 for providing one or more guiding signals to the wearer according to a further embodiment of the disclosure is shown in FIG. 9 using a wearable device such as that shown in FIGS. 5A and 5B. Rotation of the rotating body relative to the non-rotating body in a particular direction initiates the system to provide one or more guiding signals to the wearer to breath according to a breathing pattern 212. The one or more guiding signals may be haptic signals such that the wearer feels a vibration, signaling the wearer to inhale. When the vibration stops or changes, the wearer holds their breath 214. When the vibration re-starts or changes again, the wearer exhales for the duration of the vibration 216. Lastly, when the vibration signals stop or change, the wearer is guided to hold their breath prior to initiating the cycle again 218. In these embodiments, the wearer does not need to fidget the rotating body in any direction to receive the one or more guiding signals to exhale, but rather, will automatically receive guiding signals to exhale.

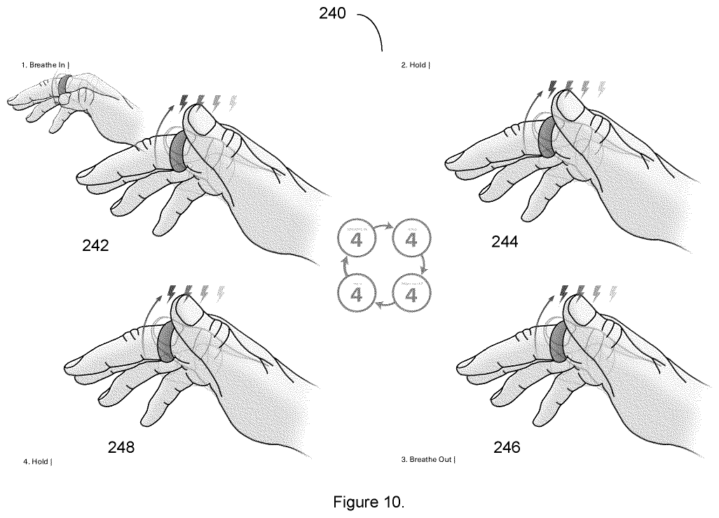

Another example of a method 240 for providing one or more guiding signals to the wearer according to a further embodiment of the disclosure is shown in FIG. 10 using a wearable device such as that shown in FIGS. 5A and 5B. Rotation of the rotating body relative to the non-rotating body in a particular direction initiates the system to provide one or more guiding signals to the wearer to breath according to a breathing pattern, while rotating the rotating body in the opposite direction allows the wearer to use the wearable device as a fidget spinner 242. The one or more guiding signals may be haptic signals such that the wearer feels a vibration, signaling the wearer to inhale. When the vibration stops or changes, the wearer holds their breath 244. When the vibration re-starts or changes again, the wearer exhales 246. Lastly, when the one or more guiding signals stop or change again, the wearer is guided to hold prior to initiating the cycle again 248. Thus, the paced haptic interaction and breathing prompt may be triggered by turning the ring in one direction, while the wearer may continue to use the wearable device as a fidget device by rotating the rotatable body in the opposite direction through all or part of the breathing pattern.

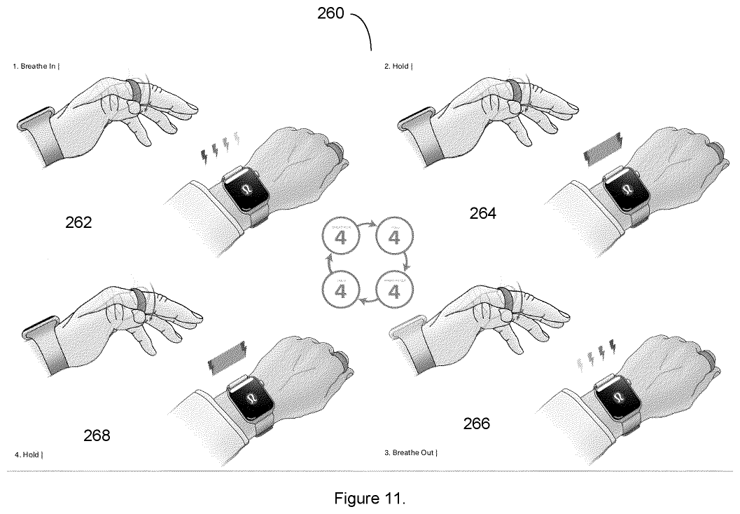

Another example of a method 260 for providing one or more guiding signals to the wearer according to a further embodiment of the disclosure is shown in FIG. 11. Rotation of the rotating body relative to the non-rotating body in a particular direction initiates the system to provide one or more guiding signals to the wearer to breath according to a breathing pattern 262. In this embodiment, the guiding element is located remotely from the wearable device 160 (FIGS. 7H and 7I), such as, for example, the guiding element is located on a smart watch. The one or more guiding signals may be haptic signals such that the wearer feels a vibration, signaling the wearer to inhale. When the wearer rotates the body 161 again, the vibration stops or changes, and the wearer holds their breath 264. When the body 161 is moved again, the wearer receives one or more guiding signals to exhale 266. Lastly, another movement of the body 161 provides one or more guiding signals to the wearer to hold prior to initiating the cycle again 268. A wearable device 270 for use in this embodiment is shown in FIG. 13. The wearable device 160 can include a position sensor (not shown) to generate one or more position signals to the controller 24 and the wearer. The method 160 can also be used with a wearable device similar to the device of FIGS. 5A and 5B that comprises a rotating body and a non-rotating body, with the guiding element being position remotely instead of within the non-rotating body.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to the present invention. Any priority document(s) and all publications, including but not limited to patents and patent applications, cited in this specification are incorporated herein by reference as if each individual publication were specifically and individually indicated to be incorporated by reference herein and as though fully set forth herein. The invention includes all embodiments and variations substantially as hereinbefore described and with reference to the examples and drawings.

1. A system for guiding breathing of a wearer through a breathing pattern, the system comprising:
a wearable device comprising a non-rotating body and a rotating body adjacent to the non-rotating body, the rotating body configured to rotate about the non-rotating body substantially frictionless or almost frictionless;
a position sensor coupled to the wearable device to generate one or more position signals;
a physiological sensor in communication with the wearer configured to detect at least one physiological parameter of the wearer and generate one or more physiological signals;
an actuator;
a guiding element in communication with the actuator and configured to provide one or more guiding signals to the wearer; and
a controller in communication with the actuator, the physiological sensor and the position sensor, the controller comprising:
an input unit for receiving the one or more physiological signals from the physiological sensor and the one or more position signals from the position sensor,
a processing unit for determining a physiological state of the wearer based on the one or more physiological signals and the one or more position signals and generating an output signal, and
an output unit for transmitting the output signal to the actuator to actuate the guiding element to provide one or more guiding signals to the wearer to guide the wearer through the breathing pattern.

2. The system of claim 1, wherein the wearable device does not include a visual display.

3. The system of claim 1, wherein the guiding element is coupled to the non-rotating body, or to the rotating body.

4. (canceled)

5. The system of claim 1, wherein the actuator is coupled to the non-rotating body.

6. The system of claim 1, wherein the one or more guiding signals are haptic signals.

7. The system of claim 6, wherein the actuator is: (a) a linear resonant actuator, the guiding element providing one or more vibrotactile guiding signals to the wearer to guide the wearer through the breathing pattern, (b) an eccentric rotating mass motor, the guiding element providing one or more vibrotactile guiding signals to the wearer to guide the wearer through the breathing pattern, (c) a voice coil actuator, the guiding element providing one or more tactile guiding signals to the wearer to guide the wearer through the breathing pattern, or (d) a piezoelectric actuator in communication with at least one of the non-rotating body or the rotating body, the guiding element providing one or more vibrotactile or pressure-tactile guiding signals to the wearer to guide the wearer through the breathing pattern.

8-10. (canceled)

11. The system of claim 1, wherein the one or more guiding signals are: (a) audio feedback signals, the guiding element comprising a speaker and a microphone, or (b) visual signals, the guiding element comprising at least one LED.

12. (canceled)

13. The system of claim 1, further comprising: (a) at least one washer positioned between the non-rotating body and the rotating body to reduce friction therebetween, or (b) a plurality of bearings positioned between the non-rotating body and the rotating body to reduce friction therebetween.

14-15. (canceled)

16. The system of claim 1, wherein the position sensor is coupled to the non-rotating body.

17. The system of claim 1, wherein one or more of a plurality of components of the position sensor is coupled to the non-rotating body and one or more of the plurality of components of the position sensor is coupled to the rotating body.

18. The system of claim 1, wherein the position sensor is a rotary encoder.

19. (canceled)

20. The system of claim 1, wherein the physiological sensor is positioned remotely from the wearable device.

21. The system of claim 1, wherein the controller is positioned remotely from the wearable device and is in communication with the wearable device by wire or wirelessly.

22. The system of claim 1, wherein the wearable device further comprises a switch.

23. (canceled)

24. The system of claim 22, wherein the switch is in communication with the controller and activation of the switch generates an input signal to the input unit that is processed by the processing unit to generate the output signal that is transmitted to the actuator to actuate the guiding element.

25. The system of claim 1, wherein the wearable device is a ring, a bracelet or a necklace.

26. The system of claim 1, wherein the controller is within a smart device.

27. (canceled)

28. A method for guiding breathing of a wearer through a pre-determined breathing pattern, the method comprising:
detecting at least one physiological parameter of the wearer with a physiological sensor in communication with the wearer and providing one or more physiological signals to an input unit of a controller;
detecting a position of a position sensor coupled to a wearable device and providing one or more position signals to the input unit of the controller, wherein the wearable device comprises a non-rotating body and a rotating body that rotates about the non-rotating body of the wearable device substantially frictionless or almost frictionless, the non-rotating body adjacent the wearer;
processing the one or more physiological signals and the one or more position signals at a processing unit of the controller to determine a physiological state of the wearer and generate an output signal;
transmitting the output signal from an output unit of the controller to an actuator;
actuating a guiding element in communication with the actuator; and
providing one or more guiding signals from the guiding element to the wearer of the wearable device to guide the wearer through the pre-determined breathing pattern.

29. The method of claim 28, wherein the input unit receives the one or more position signals from the position sensor comprising a rotary encoder, the processing unit determining the physiological state of the wearer and generating the output signal to slow down rotation of the rotating body.

30. A computer-readable medium having stored thereon computer program code configured when executed by one or more processors to cause the one or more processors to perform a method as defined in claim 28.

31-44. (canceled)
